(19) United States
(12) Patent Application Publication
(10) Pub. No.: US 2019/0015070 A1
(43) Pub. Date: Jan. 17, 2019

(54) Ultrasonic Capsule Endoscopy Device having Image-based Relative Motion Estimation

(71) Applicant: The Board of Trustees of the Leland Stanford Junior University, Palo Alto, CA (US)

(72) Inventors: Farah Memon, Sunnyvale, CA (US); Junyi Wang, Palo Alto, CA (US); Gerard Touma, Stanford, CA (US); Spyridon G. Baltsavias, Stanford, CA (US); Chienliu Chang, Palo Alto, CA (US); Morten F. Rasmussen, San Francisco, CA (US); Mohammad Amin Arbabian, San Francisco, CA (US); Butrus T. Khuri-Yakub, Palo Alto, CA (US); R. Brooke Jeffrey, JR., Los Altos Hills, CA (US); Amin Nikoozadeh, San Carlos, CA (US); Jung Woo Choe, Seoul (KR); Eric Olcott, Mountain View, CA (US); Arif Sanli Ergun, Palo Alto, CA (US)

(21) Appl. No.: 16/031,688

(22) Filed: Jul. 10, 2018

Related U.S. Application Data
(60) Provisional application No. 62/531,295, filed on Jul. 11, 2017.

(51) Int. Cl. A61B 8/12; A61B 8/00; A61B 8/08; A61B 5/00; A61B 10/00; G06T 7/73; A61B 6/00; A61M 31/00; A61N 7/02

(52) U.S. Cl. CPC A61B 8/12; G01S 15/66; A61B 8/5223; A61B 8/488; A61B 8/4263; A61B 8/56; A61B 5/0095; A61B 8/4416; A61B 10/0045; A61B 8/54; G06T 7/74; A61B 6/4057; A61M 31/002; A61N 7/022; A61B 8/0841; A61B 2560/0242; G06T 2207/10132; G06T 2207/10116; G06T 2207/10068; A61B 2010/0061; A61B 8/4254

(57) ABSTRACT

Improved localization of the capsule in acoustic capsule endoscopy is provided by using analysis of the frames of the acoustic images to deduce the relative motion of the capsule from frame to frame. This idea can be supplemented with any combination of: further localization methods; propulsion of the capsule via acoustic radiation reaction; bidirectional communication and system level feedback control; energy harvesting; photoacoustic (or x-ray acoustic) imaging; and adding therapy and/or sensor capabilities to the capsule.

### CROSS REFERENCE TO RELATED APPLICATIONS

This application claims the benefit of U.S. provisional patent application 62/531,295, filed on Jul. 11, 2017, and hereby incorporated by reference in its entirety.

### FIELD OF THE INVENTION

This invention relates to acoustic capsule endoscopy.

### BACKGROUND

Currently, thorough examination of the digestive system is available using endoscopic ultrasound (EUS), computed tomography (CT), and magnetic resonance imaging (MRI). These techniques are either harmful (CT imposes radiation), uncomfortable (EUS requires sedation and insertion of catheter) or very expensive.

### SUMMARY

The capsule ultrasound (CUS) device described herein can be a low-cost, disposable, and ingestible capsule with an ultrasound array attached to its body. After being swallowed by the patient, this capsule can allow the doctors to seamlessly examine the entire gastrointestinal (GI) tract, primarily the small intestine. The ultrasound can be used to locate abdominal disorders and lesions, find the GI wall thickness, and examine adjacent organs (such as the pancreas). To make the CUS device a more feasible alternative to aforementioned techniques, we describe additional features beyond conventional ultrasound imaging, such as localization and propulsion of the capsule.

An example of how these features and others are integrated follows: the interior of the capsule contains the imaging (plus localization, propulsion and/or energy harvesting in some embodiments) electronics that control the ultrasound transducers, power management electronics that control power distribution from battery/storage element (and harvesting transducers in some embodiments) to the rest of the electronics, battery/storage element, memory circuitry to temporarily store image/localization data, and wireless transceiver for wireless communication. In some embodiments the interior of the capsule also contains reservoir(s) for storing tissue/liquid samples or prepackaged drugs for drug delivery, and/or optical/X-ray transducers for photoacoustic or X-ray acoustic imaging. The abovementioned elements can be on different printed circuit (PC) boards, connected to each other via flexible substrate boards, or several components can be combined on one or more boards. The exterior of the capsule may include ultrasound transducers (for imaging, localization, propulsion and/or energy harvesting, depending on specific embodiment), RF antenna for wireless communication and pH electrodes/gas/pressure sensor in some embodiments. In some embodiments some of the abovementioned exterior elements such as the RF antenna can reside in the interior instead.

### BRIEF DESCRIPTION OF THE DRAWINGS

FIGS. 1A-B schematically show an embodiment of the invention.

FIGS. 2A-B show exemplary capsule configurations.

FIGS. 3A-B show further exemplary capsule configurations.

FIGS. 3C-D show field of view geometry for the capsule.

FIGS. 4A-B schematically show how several kinds of capsule motion would affect the acoustic images.

FIG. 5 schematically shows use of an anatomical landmark as a location reference.

FIGS. 6A-B show exemplary localization methods.

FIG. 7A schematically shows capsule propulsion using acoustic radiation reaction.

FIGS. 7B-G show examples of acoustic capsule propulsion.

FIGS. 8A-B schematically show exemplary communication and control for acoustic capsule endoscopy.

FIG. 9 shows harvesting externally provided acoustic energy with the capsule.

FIG. 10 shows photoacoustic or x-ray acoustic imaging by the capsule.

FIG. 11 shows addition of further capabilities to the capsule.

### DETAILED DESCRIPTION

In this description, several features of preferred embodiments are described. Embodiments of the invention can include these features individually or in any combination. The main topics in the following description are 1) relative localization, 2) absolute localization, 3) propulsion, 4) communication and control, 5) energy harvesting, 6) photoacoustic or x-ray acoustic imaging, and 7) further options and variations.

### 1) Relative Localization

While traveling inside a patient's gastrointestinal tract, the location of the capsule is important information for both disease diagnostics and capsule adaptive control. After the capsule ultrasound device captures images of the distinct layers of the small intestine as well images of the neighboring organs, it is important to acquire the location related to the images, in order to locate a specific feature, like a tumor or cyst, determine the capsule trajectory along the gastrointestinal tract, and possibly to reconstruct 3D images or a full reconstruction of the gastrointestinal tract. The location information can also serve as feedback information for an external device to control the operation mode and the frame rate of the capsule through bi-directional wireless communication.

It is helpful to distinguish between relative localization, which is identification of location changes of the capsule based on analysis of the acoustic images obtained, and absolute localization, which is identification of the position of the capsule based on comparison to external position references. We first consider relative localization.

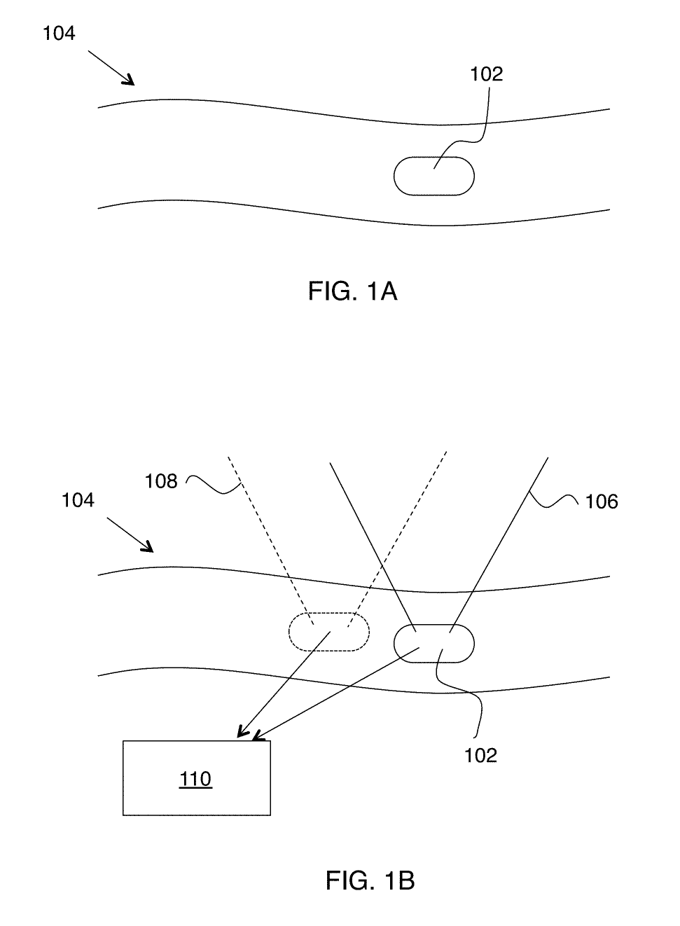

Frame-to-frame tissue movement estimation can be used to estimate and track the CUS device location and orientation as it traverses the gastrointestinal tract. FIGS. 1A-B schematically show the concept. Here FIG. 1A shows capsule 102 in GI tract 104. FIG. 1B shows how an acoustic field of view 106 can change to a new field of view 108 as the capsule moves. Since these fields of view will overlap (e.g., as shown) for typical system acoustic frame rates and capsule speeds, analysis of the acoustic images in relative location tracking subsystem 110 can provide relative motion information.

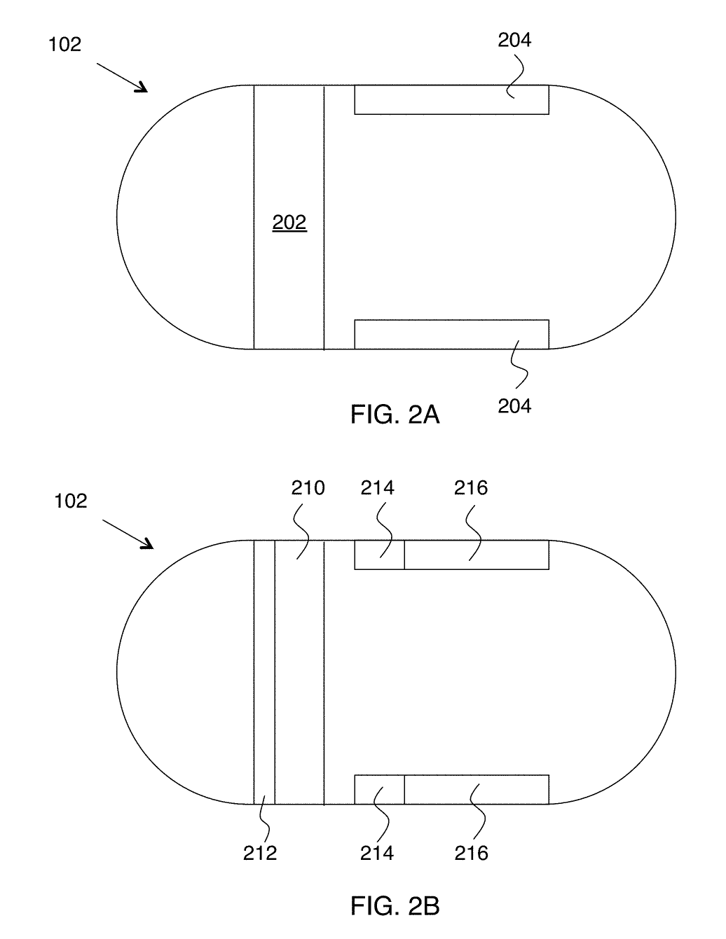

FIG. 2A is an enlarged view of an exemplary capsule 102. In this example, capsule 102 includes a cylindrical acoustic transducer array 202 disposed around the circumference of the capsule, along with one or several linear acoustic transducer arrays 204 disposed along the length of the capsule. Such linear transducer arrays are also referred to as ‘flat’ acoustic transducer arrays. This configuration allows the capsule to acquire acoustic images which are orthogonal to each other. As a result, translation in all three directions (x, y, and z-dimension) as well as rotation around all axes (x and y and z) can be calculated by cross-correlating successive images from the cylindrical and the linear arrays. This helps to track the CUS device as it propagates down the gastrointestinal tract. The frame-to-frame movement can be estimated by many different techniques, such as: local cross-correlation, speckle tracking, Doppler estimation, transverse-oscillation-method, etc.

Accordingly, an exemplary embodiment of the invention is an apparatus that includes a capsule configured to be swallowed by a patient. The capsule includes at least one diagnostic acoustic transducer array disposed on the capsule and configured to be operational while the capsule is ingested by the patient to provide acoustic images. The apparatus includes a relative location tracking subsystem configured to determine changes in relative location and/or orientation of the capsule from one frame of the acoustic images to another frame of the acoustic images using an image processing method applied to the acoustic images

The apparatus can be configured to provide a synthetic acoustic image reconstruction from two or more selected frames of the acoustic images combined with relative location and orientation of the capsule from each of the selected frames. This is analogous to synthetic aperture radar. More specifically, as the capsule is moving through the GI tract, multiple datasets, each from a new capsule position can be acquired. These datasets can be gathered from the cylindrical array and the linear array. After knowing the position of the capsule and the elements, using localization techniques, these datasets can be synthetically focused to increase both resolution and contrast in the resulting images. The synthetic focusing technique could also be used to create a full 3D image of a region of interest.

As described in greater detail below, this relative localization can be supplemented with absolute localization. In such cases, the acoustic transducer arrays on the capsule can be dual-use arrays that provide data for both imaging and the absolute location tracking, or separate location and imaging arrays can be used. FIG. 2B shows an example of the latter approach, where 210 and 212 are imaging and localization cylindrical transducer arrays, respectively. Similarly, 214 and 216 are localization and imaging linear transducer arrays.

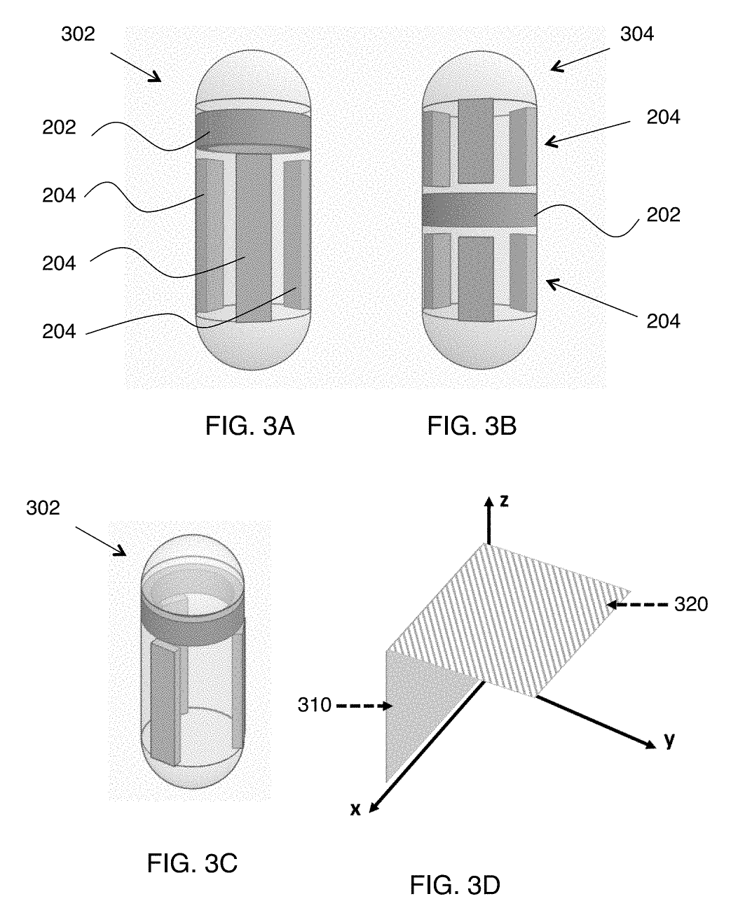

FIGS. 3A and 3B show two examples of capsules. FIG. 3A is a 3-D view of the example of FIG. 2A. FIG. 3B differs from the example of FIG. 3A in that the cylindrical transducer array 202 is disposed at the midline of the capsule, with linear acoustic arrays disposed both above and below this midline. FIGS. 3C-D schematically show the fields of view of the respective transducer arrays. Here the z-axis is taken to be along the axis of the capsule, which means that the image plane 320 of the cylindrical transducer array will be in the xy plane. The linear transducer arrays will have image planes parallel to the z axis, and one such image plane is shown as 310 on FIG. 3D.

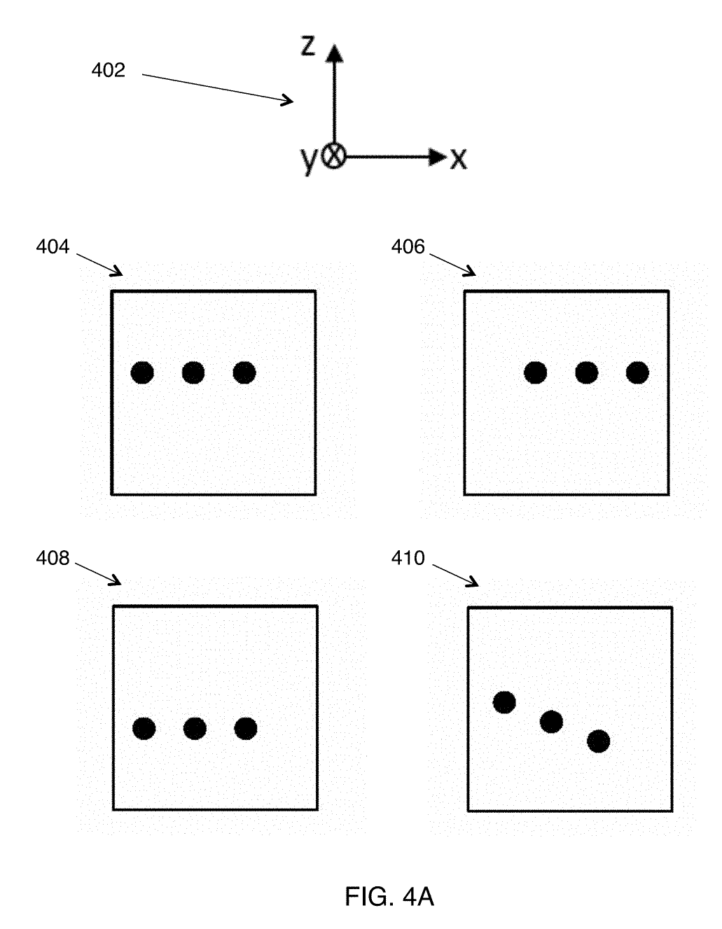

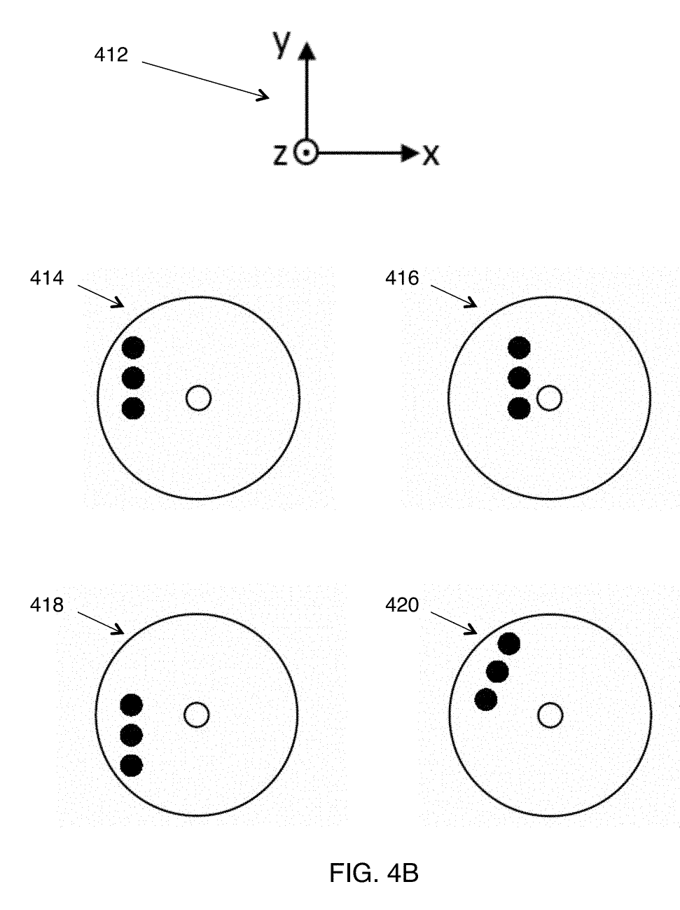

FIG. 4A schematically shows how a translation in the z and/or x directions or a rotation about the y axis can be estimated by comparing two or more acquired xz frames from the linear array. The coordinate axes 402 apply to all sample images 404, 406, 408, 410, and are also consistent with the coordinates of FIG. 3D.

Here 404 is an initial image in the xz plane from one flat array. 406 is a first example of a following image from the flat array in the xz plane. Linear translation in the x direction can be estimated by comparing the initial and the following images from the flat array. 408 is a second example of a following image from the flat array in the xz plane. Linear translation in the z direction can be estimated by comparing the initial and the following images from the flat array. 410 is a third example of a following image from the flat array in the xz plane. Rotation around the y axis can be estimated by comparing the initial and the following images from the flat array.

FIG. 4B schematically shows how a translation in the x and/or y directions or a rotation about the z axis can be estimated by comparing two or more acquired xy frames from the cylindrical array. Here the coordinate axes 412 apply to all sample images 414, 416, 418, 420, and are also consistent with the coordinates of FIG. 3D. For all sample images the inner central circle indicates the boundary of the cylindrical array and the outer circle indicates the boundary of the image.

Here 414 is an initial image in the xy plane from the cylindrical array. 416 is a first example of a following image from the cylindrical array in the xy plane. Linear translation in the x direction can be estimated by comparing the initial and the following images from the cylindrical array. 418 is a second example of a following image from the cylindrical array in the xy plane. Linear translation in the y direction can be estimated by comparing the initial and the following images from the cylindrical array. 420 is a third example of a following image from the cylindrical array in the xy plane. Rotation around the z axis can be estimated by comparing the initial and the following images from the flat array.

### 2) Absolute Localization

In other approaches for absolute localization, to determine the location of an in-vivo device, extra or external transducers (i.e. magnetic transducers) are used. These methods usually require the patients to wear external devices serving as frame coordinates, or require the patients to keep stable near a bulky instrument. These methods not only require extra devices, but also cause inconvenience for the patients. In order to eliminate these extra or external devices, we provide an ultrasonic image-guided localization and registration method, so that the location of the ultrasonic capsule could be automatically determined from the ultrasonic image generated by the capsule itself.

In addition, the location can also serve as feedback information to an external receiver and control device, and then, based on the location of the capsule, the external device could control the frame rate, propulsion direction and other operation mode through the two-way communication.

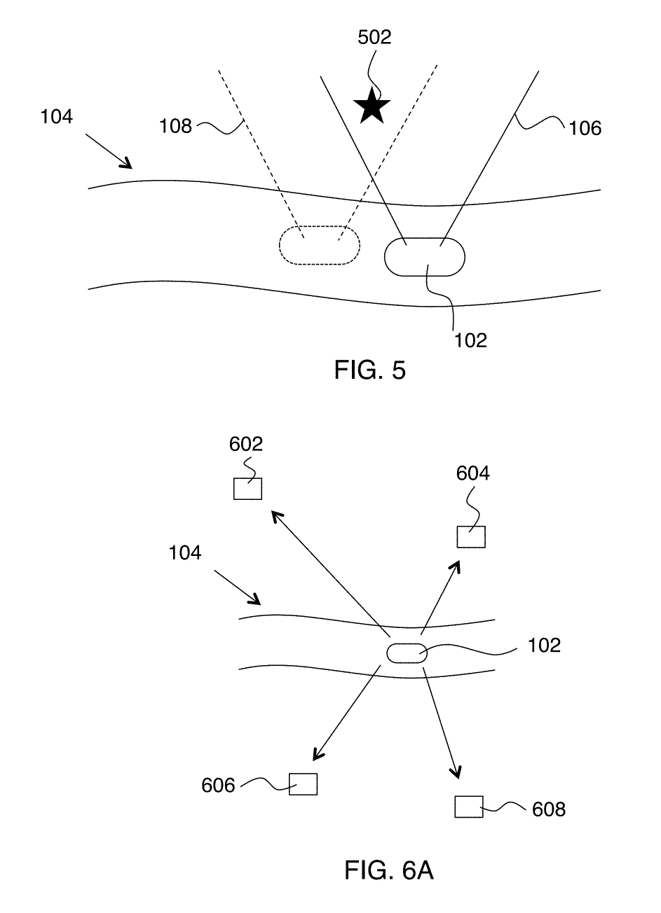

Automatic localization can be accomplished by imaging the anatomical landmarks, such as distant organs, using a cylindrical transducer array encircling around the capsule, as well as one or multiple flat linear transducer arrays along the axis direction of the capsule, as shown in FIG. 3A-B. The transducer arrays used for localization will work at modes different from the arrays used for diagnostics. The diagnostic-mode arrays require a higher frequency and better image resolution. For localization purpose, the arrays will work at localization mode, which requires a lower frequency and larger penetration depth. FIG. 5 schematically shows this concept, where 502 is the anatomical landmark.

The cylindrical transducer array and the flat linear transducer arrays along the long axis direction of the capsule have a penetration depth of up to 10 cm. The images generated by these transducer arrays would show anatomical landmarks around the GI tract, therefore, by calculating the relative distance to the anatomical landmarks according to the captured image, the location of the capsule device can be determined as it travels the digestive system. Meanwhile, because the 360 degrees ultrasound scans of the cylindrical transducer array always start from the same elements, these ultrasound images can provide information of the capsule rotation around its axis. The linear transducer arrays can generate images at a higher frame rate and the images can be used to trace the capsule movement. Because of the high frame rate, the peristalsis movement can be traced correctly. As indicated above, a cross-correlation method can be used to calculate the relative movement between two successive images captured by the same transducer array, so that we can trace the capsule rotation using the images acquired by the cylindrical array and calculate the longitudinal capsule movement using the images of the flat arrays. Based on the capsule rotation and movement, the image registration can be performed by reconstructing the images into the same coordinate system, so that the 3D image along the GI tract can be eventually reconstructed.

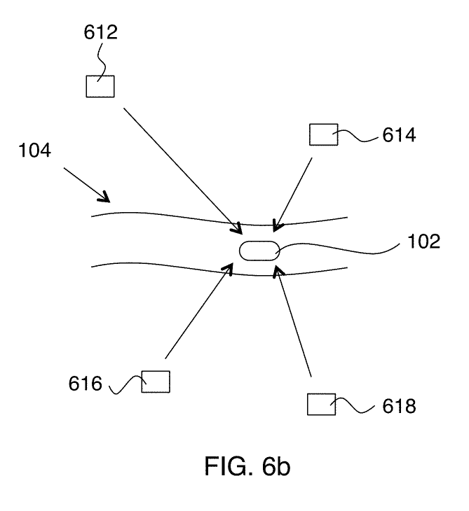

Localization can also be accomplished using another technique that involves putting several external airborne ultrasound transducers in the room where the patient stays (such as the on the chairs, on the bed, on the wall) and carry out trilateration. FIG. 6A shows an example, where 602, 604, 606, 608 are the external transducers operating in receive mode. These transducers will operate in the receive mode at a low frequency (i.e., 100 kHz) and listen to the ultrasound signals emitted by the corresponding low frequency transducers in the capsule. Knowing the time of the emission of the signal by the transducers in the capsule and the time of pulse detection by the transducers located outside of the body, the time-of-flight algorithm can be used to determine the location of the capsule inside the body. Or in the opposite way, the transducers located outside the body can be used as transmitters and the received signals by the transducer on the capsule is used to localize the capsule inside the body, using various algorithms such as how global positioning system (GPS) works. FIG. 6B shows an example, where 612, 614, 616, 618 are the external transducers operating in receive mode.

The RF antenna and external receiver device, as described in greater detail below, can also contribute time-of-flight information, which can improve the accuracy of the capsule location calculation. This RF system can contain a pulsed-based duty-cycled transmitter running at one or more frequencies. Time of flight and trilateration techniques can be used to locate the transmitter (capsule) with respect to an array (3 or more) of external receivers. A differential time of flight method could also be used. Here, multiple transmitters (two or more) on two sides of the capsule transmit short RF pulses. The difference in time of arrival can be used to locate the capsule. The RF frequency will be selected as a compromise between tissue losses and antenna efficiency (leading to SNR constraints) vs. D-TOF accuracy. In all these systems the preferred pulse width resides in the 100 ps to 10 ns range. Chemical environment information (e.g. pH) acquired by additional sensors could serve as another indicator for localization.

### 3) Propulsion

During wireless capsule endoscopy, the capsule typically travels in the GI tract passively via peristalsis. This imposes several limitations on the imaging performance of the capsule, as well as its capabilities to perform other medical tasks such as drug delivery and biopsy. These limitations include: Higher percentage of missed findings in the screening/diagnosing process; Inability to stabilize the capsule to allow for treatment delivery; Inability to direct the capsule toward areas of interest (e.g. pathologies) to acquire images, treatment delivery, biopsy, etc.; and Lower effective field of view.

The ability to propel the capsule inside the GI tract provides several benefits that could allow exploring new features and new capabilities. The ability to stabilize the capsule in the GI tract improves its diagnostic and therapeutic capabilities by allowing one to control the number of acquired images of the same area of interest.

Propulsion could also be used to position and orient the capsule in such a way to enable imaging specific internal organs with either ultrasound imaging, photo-acoustic imaging, X-ray acoustic imaging, or any other imaging modality. Another benefit is to provide the capsule with adaptability to the different anatomies of the various organs in the GI tract.

Propulsion can also help in revisiting certain locations of abnormalities, and to control the duration the capsule stays inside the GI tract. This can help control the energy consumed, and the examination time.

To propel the capsule in the GI tract, the transducer array can be wrapped around the capsule to generate ultrasonic waves that can drive the capsule in any desired direction. In one implementation, the array can be used as an interdigital transducer (IDT) to generate surface acoustic waves (SAW). The direction of these waves is such that it opposes the desired direction of propulsion, and their acoustic pressure dictates the speed at which the capsule travels. Another implementation uses the transducer array as a phased array. To propel in a particular direction, a subset of the array elements generates a focused ultrasound beam in the opposite direction. A subset of transducer elements can generate focused ultrasound beams with variable directivity, and intensity.

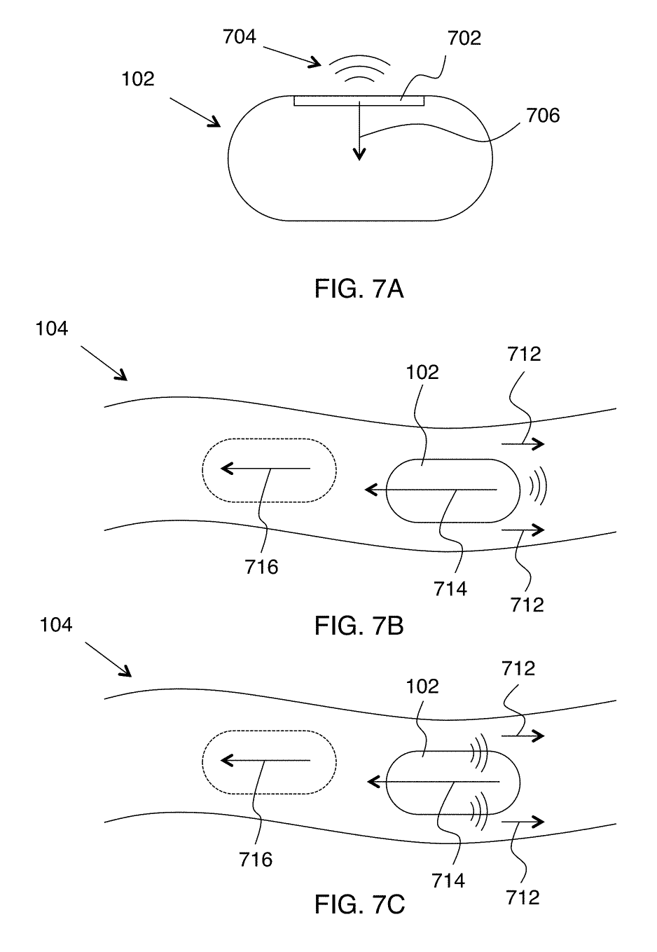

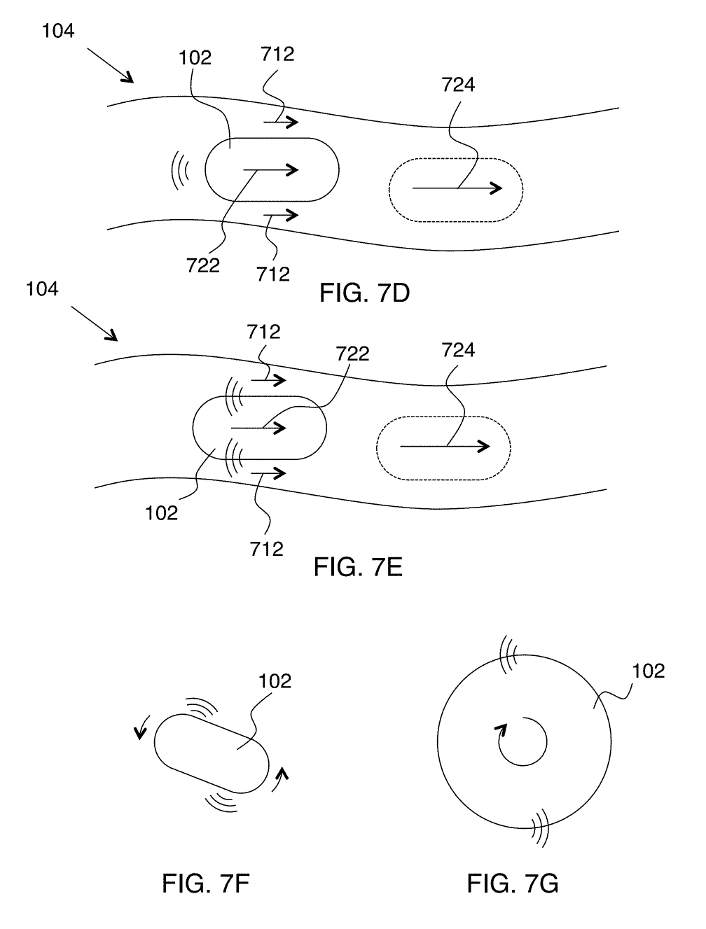

More specifically, the propulsion concept considered here is that capsule be self-propelled while ingested by an acoustic radiation reaction force on the capsule due to emission of acoustic energy by the capsule. FIG. 7A shows this concept. Here 102 is the capsule, 702 is the acoustic transducer, 704 is emitted acoustic radiation and 706 is the radiation reaction force on the capsule. As suggested by FIG. 7A, the reaction force is generally in the opposite direction to any directional acoustic radiation. Note that this radiation reaction force does not depend on how the contents of the GI tract move in response to the acoustic radiation, in contrast to other approaches where the induced flow of GI tract contents is the propulsion mechanism.

FIGS. 7B and 7C schematically show propulsion of capsule 102 backward relative to a peristalsis 712 for the phased array and IDT cases respectively. Here the net motion 716 is the superposition of peristalsis 712 and radiation reaction 714. Similarly, FIGS. 7D and 7E schematically show propulsion of capsule 102 forward relative to peristalsis 712 for the phased array and IDT cases respectively. Here the net motion 724 is the superposition of peristalsis 712 and radiation reaction 722. FIG. 7F shows how capsule 102 can be rotated about its midline by radiation reaction forces. FIG. 7G (which is an end view) shows how capsule 102 can be rotated about its axis by radiation reaction forces.

In addition to propelling the capsule backward, forward, or in any direction that changes its current location, the capsule can also be rotated in both directions around its axis. This, for example, can help improve the resolution of the acquired images by rotating the capsule by a distance smaller than the transducer elements pitch, which effectively provides higher resolution. Another benefit of rotating the capsule around its axis is to create better contact with the surrounding tissue/organ.

Acoustic propulsion provides significant advantages compared to conventional propulsion approaches. Several capsule designs use magnetic fields to rotate the capsule and translate its location by applying an external magnetic field that interacts with another metal or magnetic body placed inside the capsule body. This requires extra area, external requirement, and of course, external monitoring. Several other implementations rely on using robotic parts to control the position of the capsule and push it in a particular direction. These types of implementations are highly invasive, consume a lot of power, are complex to design, and require a large additional area. Another implementation uses electrodes to electrically stimulate the organ wall and cause it to contract. This emulates the effect of peristalsis, and therefore pushes the capsule forward. This technique is highly invasive as well.

Compared to the current implementations, the design of this work is non-invasive. It uses acoustic signals to propel the capsule, without requiring any contact with the biological tissue. This implementation also does not require any additional parts attached to the capsule, and no external devices. This reduces the design complexity since the imaging transducer array that is already wrapped around the capsule can also be used for propulsion, and does not require any contact with the surrounding tissue which can typically cause contaminations, and other unwanted effects.

### 4) Communication and Control

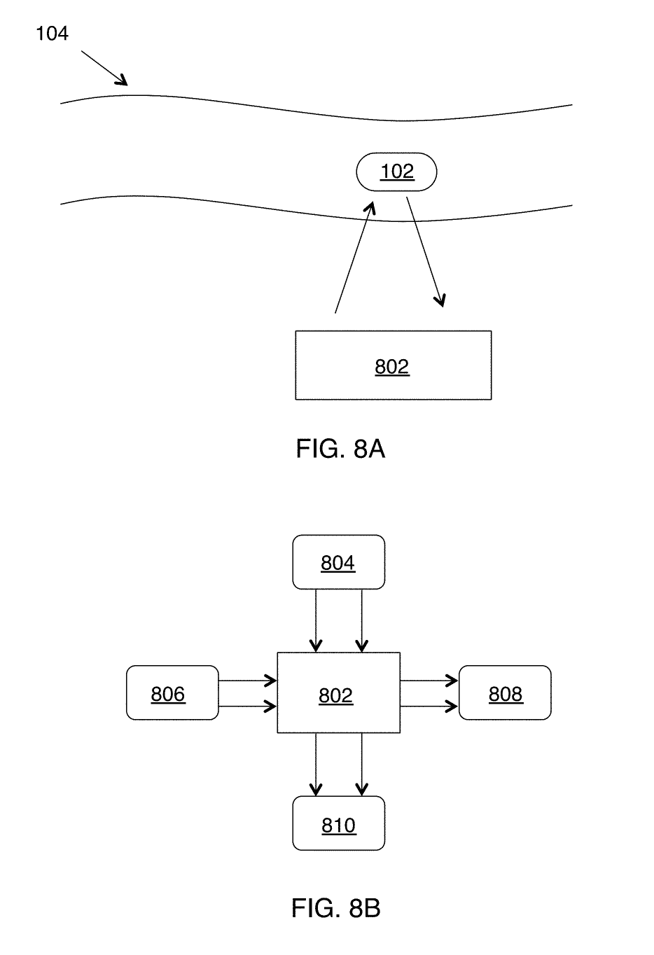

FIG. 8A schematically shows closed loop control of capsule 102. Here controller 802 is in bidirectional communication with capsule 102. This link can be an acoustic link and/or a wireless electromagnetic link, as described in more detail below. With such a link in place, feedback control can be used to control one or more operational parameters of the capsule. FIG. 8B shows some further details relating to such configurations. Here input block 804 is part of the user interface and includes items such as user input (both patient and medical professional), desired capsule location, desired capsule mode of operation (e.g., ‘enhanced imaging mode’), etc. Input block 806 is the uplink from the capsule, and can include parameters such as capsule orientation (pitch, roll, yaw), location, velocity, pressure, system status, remaining energy, etc. Output block 808 is the downlink to the capsule, and can include parameters such as propulsion force (magnitude and direction), image focus depth, image frame rate, etc. Output block 810 is part of the use interface and can include items such as system status, remaining capsule energy, capsule location, capsule orientation, target location, target orientation, selected mode of operation etc. Further details on communication/control in preferred embodiments follow.

An uplink wireless connection (from the capsule to an external to the body device) can serve multiple purposes and is often important for ingestible capsule applications. The uplink can be used for collection of sensed data, such as images/video of an optical/ultrasound capsule. This communication can eliminate the need for the collection of the capsule itself, allowing it to be disposable. The uplink communication can furthermore be used for collection of other sensed data including but not limited to: capsule position, orientation, chemical environment properties, remaining battery lifetime, system status and component performance, biomarkers such as proteins or DNA, or neural/muscle electrical activity, etc. In certain embodiments, sensors can be integrated into the ingestible capsule to monitor the condition of the body in terms of temperature, heart rate, respiration rate, pH and the collected data from each sensor can also be sent wirelessly to an external controller through the uplink. The combination of these parameters determines the new programmed state of the capsule in terms of imaging and sensing conditions.

This information can be then processed externally in a less resource-limited device and provide user-friendly data to a user or medical professional. This data can then enable accurate disease diagnosis in itself or as part of other collected patient data and diagnostic measurements. Additionally, it can be used for the issuing of commands to be sent to the capsule, to dynamically change operational parameters including but not limited to: imaging transducer delay, imaging focus depth, imaging direction, imaging frame rate, propulsion pressure/angle of rotation/rotational velocity, chemical parameter to be sensed, uplink transmitter output power, uplink data rate, etc. These commands can be communicated from the external device to the capsule through a downlink wireless connection, in a similar way the previously described uplink functions.

The uplink and downlink wireless connections can further form a bi-directional, closed loop feedback system. This can enable automatic capsule parameter control through sensed data, by performing analysis of the data and finding optimal sets of output parameters in an external device processor or look-up table, such that a desired operation of the capsule is tuned to adapt to changes in the traversed gastrointestinal tract anatomy, and specifics of physical orientation, position, chemical environment, user body condition, etc. This closed loop feedback can for example be used to enhance imaging of a particular body region such as pancreas for the diagnosis of pancreatic cancer or GI tract wall for the detection of polyps, once the capsule location and field of view meets certain predefined conditions, through motion and frame rate control. In certain embodiments, this closed loop feedback can enable the specific motion in response to changes in the pH or specific reactions along the GI tract, for localized drug treatment. The implementation of such a bi-directional link thus constitutes a very important system feature to overcome uncontrollable parameters such as patient to patient variations and anatomical differences in the gastrointestinal system.

Communication between the capsule and an external device for an uplink can be achieved by electromagnetic (EM) radiation in radio frequencies (RF). In such embodiments, one or more antennas are included inside or outside the capsule and transmit EM waves through the body, to be received by one or more antennas external and in proximity to the body. The antennas are driven by amplifier circuits as part of transmitter circuits to allow enough power to be transmitted such that information is received with sufficient signal to noise ratio (SNR) at the receiver to meet a fidelity requirement. In certain embodiments the transmitter encodes data via digital modulation, such as frequency shift keying (FSK), and the amplifier provides a constant amplitude, varying frequency waveform generated by oscillator based circuits, that is encoded by the baseband data, which could be a digital representation of captured ultrasound or optical images, location/orientation information, system status and performance information, body environment physical indicators (temperature, heart rate, respiration rate), chemical environment (e.g. pH), etc. In some embodiments, the operation of the transmit circuitry is variably active and inactive (duty-cycled), and/or time-interleaved with other capsule operations, including but not limited to imaging, chemical sensing, chemical sampling, to meet certain data rate requirements or as part of energy saving schemes. In some embodiments where collected data by other components of the capsule, such as the imager, are available at a rate higher than the possible data rate of the transmitter circuitry, a memory storage element, such as a RAM (random access memory), or FIFO (first-in, first-out) element can be used to temporarily buffer the sampled data until it is ready to be transmitted by the transmitter.

Similarly, for downlink communication, between the external device and the capsule system, the communication may happen by means of EM radiation through one or more antennas on/in the capsule and one or more antennas outside/on the body. The transmitter circuitry on the outside device similarly may include a power amplifier, data encoder, oscillator based frequency generators, etc. It may also include a multiple number of antennas as part of a phased array to focus radiation to the capsule target. Due to the data rate of downlink commands for biomedical devices being commonly lower than the data rate of uplink, ultrasound data transmission which typically possesses a smaller bandwidth can also be utilized, for a higher link efficiency, thus forming a hybrid RF/US bi directional link. In embodiments where the capsule already uses such elements for other applications, e.g. ultrasound imaging or propulsion, time interleaving schemes can be used to alternately perform these operations with communication. E.g. use the ultrasound transducers for 10 ms to transmit a pulse and receive a scan to form an image, 230 ms for transmitting uplink data, and 10 ms for receiving downlink commands, for a total of 250 ms repeated throughout system lifetime or until a command to change operational modes is issued. For downlink communication, similar data encoding as uplink can be used, with modulation schemes such as amplitude shift keying (ASK), on off keying (OOK), FSK, etc. The receiver inside the capsule possesses circuitry to perform data recovery of commands, interpretation, and depending on the command, execution of functions such as sensor actuation, system parameter modification, e.g. rotation, pressure, imaging focus, frame rate, RF output power, data rate, drug release amount, image slice selection, chemical parameter to be sensed, etc.

With a combination of uplink and downlink, closed loop feedback can be achieved in the following fashion. Periodically/at desired time intervals, a processing unit/controller in the external device takes as inputs the received parameters (location, orientation, etc.) via the uplink. It may then combine these inputs with predefined inputs from a user/clinician relating to the operational mode of interest (e.g. ‘enhanced imaging of pancreas’, or ‘power minimization’), and via a function, map the inputs to outputs in an optimal way. The mapping may occur via look up tables and predefined relationships between inputs to outputs, or in other ways including deep learning and neural networks. The outputs correspond to system parameters to be tuned/changed in the capsule, (e.g. imaging transducer delay, imaging focus depth, imaging direction, imaging frame rate, propulsion pressure/angle of rotation/rotational velocity, drug release amount, chemical parameter to be sensed, uplink transmitter output power, uplink data rate) and are transmitted via the downlink. They are then decoded by the receiver circuitry in the capsule, interpreted and acted upon to result in a desired function such as ‘stop movement’, ‘rotate capsule’, ‘release 10 ug of drug’, etc. When errors between the resulting action and desired action exist, the processing unit/controller at the external device can infer them from the inputs resulting from the uplink, and include them in the optimal output calculation for the next set of outputs. This closed loop feedback results in dynamically adapting system parameters that achieve a desired predefined or dynamically redefined outcome relating to a capsule system operation in the gastrointestinal tract.

### 5) Energy Harvesting

The purpose of harvesting power from sources external to the capsule is to ease constraints introduced by electrochemical batteries, which are commonly used for powering electronic capsules. These constraints include size, weight, biocompatibility, lifetime and application-specific capabilities of the capsule system, and together they may significantly limit the clinical usefulness of conventional capsules.

Power harvesting can be used to recharge a battery. Current batteries such as silver oxide batteries require a long time to charge. However, new breakthroughs such as batteries with organic contacts have much faster charging times (in the order of seconds), are environmental friendly as they do not contain harmful heavy metals, and can be fabricated in small sizes. Other breakthroughs include Lithium-Sulfur (Li—S) batteries which are reported to have 3-5 times higher capacity than current batteries. This would allow implementing new features, and/or improve the current functionalities of the system such as higher frame rate, which leads to a batter image quality, and more flexibility and torque during propulsion.

For a fixed desired lifetime, power harvesting to recharge a battery allows for a smaller capacity battery to be used inside the capsule, thereby a) reducing the capsule weight, b) reducing the capsule dimensions and making it easier to ingest, c) freeing up space to ease the physical design of other capsule components, e.g. antenna for wireless communication, actuators, interconnects etc., or c) freeing up space to allow for the implementation of additional features and inclusion of other components, e.g. drug reservoirs for drug release applications. Alternatively, for a fixed battery size/capacity, harvesting to recharge the battery can a) relax the electrical requirements (e.g. peak/average power dissipation) of the capsule system and subsystems, e.g. imager, wireless transceiver, etc., b) extend the lifetime of the device allowing for longer operation and e.g. traversal of a larger portion of the gastrointestinal tract, or c) allow for the implementation of additional features that would require additional power. A particular feature that could be important for achieving clinical significance in capsule systems is propulsion, and power harvesting to recharge a battery could be used to make this feature possible by providing the additional power needed.

Power harvesting can also be used to eliminate the need for an electrochemical battery. With a temporary storage mechanism such a capacitor, continuous or periodic harvesting can similarly a) reduce the capsule weight, b) reduce the capsule dimensions, c) free up space to ease the physical design of other components and allow inclusion of extra components, d) extend the operational lifetime of the system, all crucial parameters of a capsule system. Additionally, it can eliminate a major source of toxicity and a concern of biocompatibility that many capsule systems face.

Energy harvesting can primarily be achieved through ambient harvesting, by generating electricity from the surrounding environment and within the human body, or through wireless power transfer. The ability to scavenge energy without an external power unit makes an ambient harvesting approach attractive for long-term autonomous operation, however with wireless power transfer, higher power levels can be more consistently and reliably achieved in a small form factor. A technique to perform wireless power transfer is via ultrasound waves. For capsule applications ultrasound powering could have many advantages: The wavelength of ultrasound traveling in the body is on the order of millimeters for a frequency range of 1-10 MHz (the speed of sound in tissue is about 1500 m/s), signifying that acoustic energy can be efficiently focused to specific capsule locations. Additionally, the low attenuation of human tissue (in the order of 1 dB/(cm MHz)) allows the power link from transmitter to receiver to be highly efficient compared to other techniques.

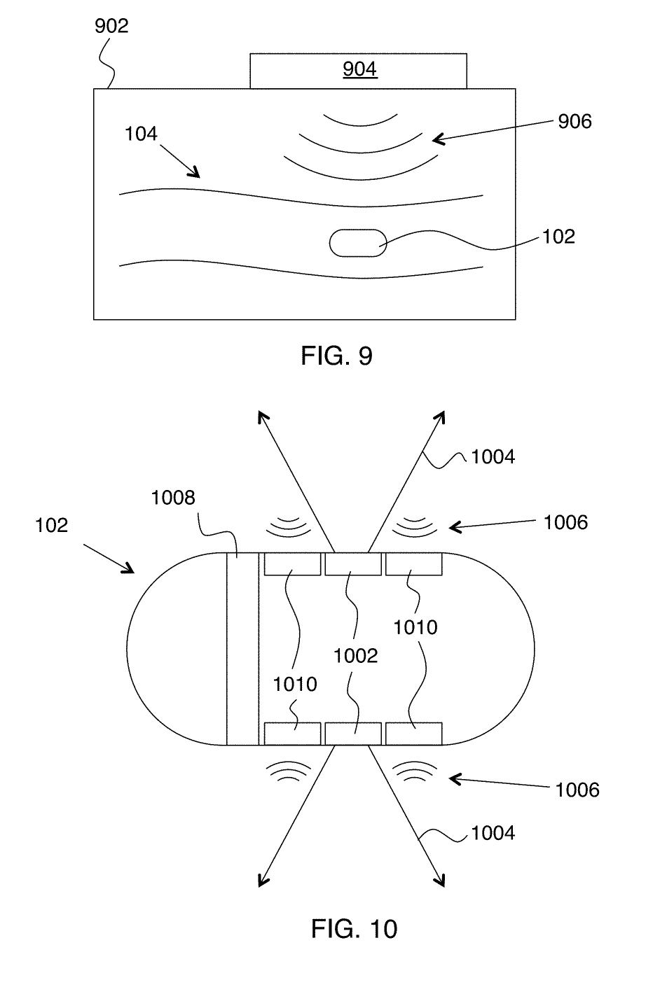

FIG. 9 schematically shows this approach, where a transducer or transducer array 904 placed on the skin of patient 902 directs acoustic energy 906 to capsule 102 which can harvest this energy with its acoustic transducers.

Ultrasound powering could be achieved by including one or more ultrasonic transducers (piezoelectric elements, capacitive micromachined ultrasonic transducers or other) on the external to the body device, in contact with the body, transmitting and beamforming periodically/at predefined time intervals ultrasonic waves to be received by one or more ultrasonic transducers in or on the capsule. The capsule circuitry may include a power recovery chain connected to the receive transducers that rectifies and regulates the incoming waves, then stores the resulting DC energy onto a storage element such as a capacitor or battery, which may be lithium-ion, zinc-oxide, lithium-sulfur for higher charging rate and capacity, or other types.

In embodiments where the capsule already includes ultrasonic elements for imaging/propulsion, the same elements could be used for wireless harvesting in a time interleaved fashion. Depending on the desired profile of powering, e.g., continuous powering or battery recharging, interleaving can be in short, e.g., 500 ms imaging/propulsion and 500 ms energy harvesting, or long, e.g., 1 hour imaging, 1 hour recharging. The implementation of a circular transducer array surrounding the capsule as well as a perpendicular linear array could have the additional benefit of providing relatively orientation independent harvesting capability, a fundamental feature for a capsule that is continuously in motion in the gastrointestinal tract.

### 6) Photoacoustic or X-Ray Acoustic Imaging

In addition to ultrasonic imaging, the capsule could also be capable of performing photoacoustic imaging. Multiple light-emitting-diodes (LEDs) and/or laser diodes can be located on the capsule in conjunction with the array being wrapped around its body. The light from the LEDs will illuminate the tissue, triggering it to undergo expansion and contraction and ultimately create ultrasound waves, which will be sensed by the cylindrical array. This technique will be valuable in detecting the biochemical nature of the digestive system. For example, cancerous regions emit a high photoacoustic signal because of the abundance of blood vessels. Hence, the photoacoustic and the ultrasound data can both be used to provide a comprehensive picture of the digestive system. In addition to photoacoustic imaging, the capsule can be used to perform x-ray acoustic imaging using a source inside the capsule that transmits X-ray waves into the medium, causing the medium to experience a change in temperature and emit pressure waves. These pressure waves can be detected using the transducers on the exterior of the capsule and processed to render x-ray acoustic images.

FIG. 10 shows this concept for both the photoacoustic and X-ray acoustic cases. Here capsule 102 includes sources 1002 (optical or X-ray) which emit radiation 1004. Absorption of radiation 1004 in the body leads to acoustic signals 1006 which are received by transducers 1008 (cylindrical) and 1010 (linear) to provide photoacoustic imaging or x-ray acoustic imaging according to the source type employed.

### 7) Further Options and Variations

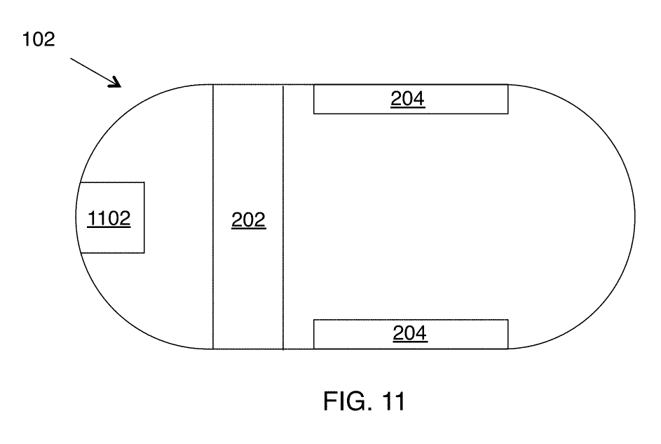

Capsule 102 can also be enhanced with various other functions. FIG. 11 shows this idea in general, where module 1102 is added to capsule 102. One option for module 1102 is a therapy module, which can be configured to provide therapy such as high intensity focused ultrasound and drug delivery. The ultrasound transducer on the capsule can be excited via a CW wave to provide HIFU (high intensity focused ultrasound) at a desired location. Propulsion techniques (described above) can be used to situate the transducer at a certain location so that the HIFU is applied at a region of interest. In addition, closed-loop feedback can be used to trigger HIFU once a cancerous lesion is detection in the GI tract. During HIFU, imaging can be done with the same array or a different array to ensure that the HIFU is being implemented correctly. In addition, the capsule can also deliver drugs at a cancerous region. The drug can be placed inside a reservoir within the capsule and released once a cancer/lesion is detected. The ultrasound array can be used to facilitate the release of the drug using cavitation and/or radiation force.

Another option for module 1102 is a sensor/sampling module, capable of functions like: fluid sampling, tissue sampling, and ambient environmental sensing. In addition to the ultrasonic imaging, the capsule could also operate as a chemistry lab by integrating other sensors, such as pH sensor, chemical gas sensors and pressure sensor, inside the capsule or at the surface of the capsule. At the same time, a MEMS micromotor could also be integrated in the capsule to control MEMS window and tips movement, so that the biological samples (tissue sample or liquid sample) from the patient's GI tract could be acquired and kept inside the capsule. These samples could be used for future analysis.

1. Apparatus comprising:
a capsule configured to be swallowed by a patient;
at least one diagnostic acoustic transducer array disposed on the capsule and configured to be operational while the capsule is ingested by the patient to provide acoustic images;
a relative location tracking subsystem configured to determine changes in relative location and/or orientation of the capsule from one frame of the acoustic images to another frame of the acoustic images using an image processing method applied to the acoustic images.

2. The apparatus of claim 1, wherein the image processing method is selected from the group consisting of: local cross-correlation, speckle tracking, Doppler estimation, and transverse oscillation method.

3. The apparatus of claim 1, wherein the capsule is configured to be self-propelled while ingested by an acoustic radiation reaction force on the capsule due to emission of acoustic energy by the capsule.

4. The apparatus of claim 1, wherein the apparatus is configured to provide a synthetic acoustic image reconstruction from two or more selected frames of the acoustic images combined with relative location and orientation of the capsule from each of the selected frames.

5. The apparatus of claim 1, wherein the apparatus is further configured to provide absolute location tracking.

6. The apparatus of claim 5, wherein the absolute location tracking employs one or more anatomical landmarks as location references.

7. The apparatus of claim 5, further comprising one or more acoustic transmitters disposed outside the patient, wherein the absolute location tracking includes determining a location of the capsule based on signals received from the acoustic transmitters at the capsule.

8. The apparatus of claim 5, further comprising one or more acoustic receivers disposed outside the patient, wherein the absolute location tracking includes determining a location of the capsule based on signals received from the capsule at the acoustic receivers.

9. The apparatus of claim 5, further comprising at least one localization acoustic transducer array disposed on the capsule and configured to be operational while the capsule is ingested by the patient to provide data for the absolute location tracking.

10. The apparatus of claim 5, wherein the at least one diagnostic acoustic transducer array is configured as a dual-use array further configured to be operational while the capsule is ingested by the patient to provide data for the absolute location tracking.

11. The apparatus of claim 1, wherein the capsule is configured to be powered while ingested by energy harvesting.

12. The apparatus of claim 1, further comprising an optical source disposed on the capsule, wherein the at least one diagnostic acoustic transducer array is configured to provide photoacoustic imaging of acoustic radiation generated in the patient by absorption of light from the optical source.

13. The apparatus of claim 1, further comprising an X-ray emitting source disposed on the capsule, wherein at least one diagnostic acoustic transducer array is configured to provide x-ray acoustic imaging of acoustic radiation generated in the patient by absorption of X-rays.

14. The apparatus of claim 1, further comprising a therapy module disposed on the capsule and configured to provide a targeted therapy selected from the group consisting of: high intensity focused ultrasound and drug delivery.

15. The apparatus of claim 1, further comprising a sensor/sampling module disposed on the capsule and configured to provide a functionality selected from the group consisting of: fluid sampling, tissue sampling, and ambient environmental sensing.

16. The apparatus of claim 1, further comprising a feedback control system configured to control one or more operational parameters of the capsule.

17. The apparatus of claim 1, further comprising an acoustic and/or wireless electromagnetic bidirectional communication link between the capsule and a component external to the patient.
